(19) United States
(12) Patent Application Publication
(10) Pub. No.: US 2007/0291897 A1
(43) Pub. Date: Dec. 20, 2007

(54) Mammography Device Having an Adjustable-Height Platform

(76) Inventors: Martin Ramsauer, Pyrbaum (DE); Peter Tichy, Uttenreuth (DE)

Correspondence Address: BRINKS HOFER GILSON & LIONE, P.O. BOX 10395, CHICAGO, IL, 60610, US

(21) Appl. No.: 11/660,018

(22) PCT Filed: Sep. 5, 2005
(86) PCT No.: PCT/EP05/54346

§ 371 Date: Feb. 8, 2007

(30) Foreign Application Priority Data
Sep. 9, 2004 (DE) 10 2004 043 739.4

(51) Int. Cl. A61B 6/04

(52) U.S. Cl. 378/37

(57) ABSTRACT

A mammography device is provided. The mammography device includes an adjustable-height platform, and a transmitter that generates an optical signal. The optical signal is dependent on the height of the platform and is projectable onto a patient standing in front of the platform.

The present patent document is a §371 continuation of PCT Application Serial Number PCT/EP2005/054346, filed Sep. 5, 2005, designating the United States, which is hereby incorporated by reference. This patent document also claims the benefit of DE 10 2004 043 739.4, filed Sep. 9, 2004.

### BACKGROUND

The present embodiments relate to a mammography device having an adjustable-height platform.

Mammography is a standard method of diagnosis, especially for precautionary examination of breast cancer. Mammography devices are constructed so that the breast to be examined, before the X-ray is taken, is placed on an object platform of the device. This placement initially requires adjusting the object platform to the height of the patient's breast. This height adjustment is done while the patient is standing in front of the mammography device. In the usual mammography devices, this height adjustment has to be estimated with the naked eye, which is often unsuccessful until multiple corrections have been made.

### SUMMARY

The present embodiments may obviate one or more of the limitations or drawbacks inherent in the related art. For example, in one embodiment, a mammography device is capable of making a precise, fast adaptation of the height of the platform to the examination region of the patient.

In one embodiment, a mammography device includes an adjustable-height platform, and a transmitter that generates an optical signal. The optical signal being dependent on the height of the platform and being projectable onto a patient standing in front of the platform.

In one embodiment, an optical signal, which is dependent on the applicable height of the object platform, is projected onto the patient. The height of the object platform can be reproduced onto the body of the patient. The height of the object platform may be adapted exactly because of the improved aiming capability in comparison to estimation with the naked eye. The object platform may be adapted with an accelerated work sequence. Multiple readjustments of the height of the object platform, which are tedious for the mammography device operator and stressful to the patient, are avoidable.

In one embodiment, the optical signal includes a beam of light. The projection can be achieved by using the beam of light as the optical signal. A sharp and recognizable projection is obtained when a laser beam is employed.

In another embodiment, a mammography device includes an adjustable-height object platform and a transmitter that generates an optical signal. The optical signal is dependent on the height of the platform and is projectable onto a patient standing in front of the platform. The optical signal is reflectable from the patient when the height of an examination area coincides with the height of the object platform.

In one embodiment, the mammography device includes a sensor that is sensitive to the optical signal. The sensor is structurally combined (coupled) with the object platform. In one embodiment, the height of the object platform is automatically adjusted as a function of the reflected signal measured by the sensor.

German Patent Disclosure DE 103 05 640 A1 discloses a mammography device with an ultrasound measuring sensor and with a position sensor of the ultrasound measuring sensor. The position sensor is a remote sensor for contactless determination of the position of the ultrasound measuring sensor by an optical signal. Different X-ray and ultrasound exposures are performed without having to move the patient and re-adapting the device. The position is ascertained with the position sensor. Once the position of the measuring sensor is known it is possible to process an echogram, which is detectable with the ultrasound measuring sensor. The echogram matches an X-ray image that can be made with the mammography device.

### BRIEF DESCRIPTION OF THE DRAWINGS

FIG. 1 is a side view of one embodiment of a mammography device with a object platform, a transmitter, and a patient standing in front of the object platform before the height of the object platform is adjusted;

FIG. 2 shows the arrangement of FIG. 1 once the height of the object platform has been adjusted;

FIG. 3 is a side view of one embodiment of a mammography device before the height of the object platform has been adjusted to a suitable height.

FIG. 4 is a side view of the embodiment of FIG. 3 of a mammography device once the height of the object platform has been adjusted to a suitable height.

### DETAILED DESCRIPTION

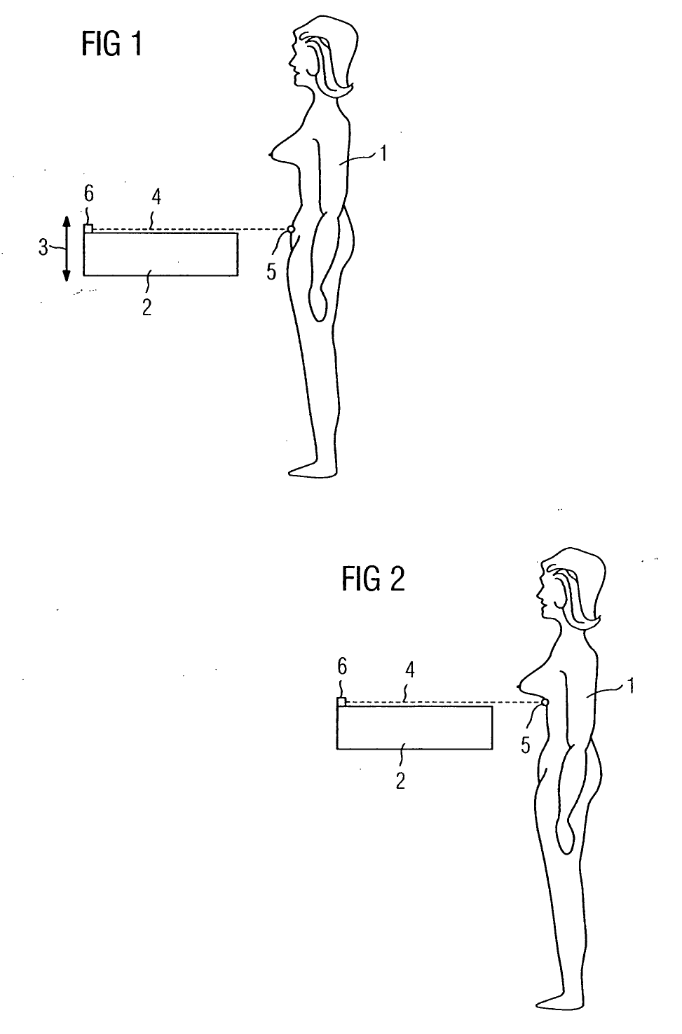

FIG. 1 is a side view of a patient 1 standing in front of an object platform 2. The height of the object platform 2 is adjustable, in the direction represented by an arrow 3, relative to the examination region of the patient 1. For the height adjustment, an optical signal 4 that corresponds to the applicable height of the object platform 2 can be projected onto the patient 1. As a result of the projection 5 of the optical signal 4, the height of the object platform 2 can be reproduced on the body of the patient 1, so that the object platform 2 is adjustable to the height, shown in FIG. 2, that is suitable for the examination. In one embodiment, as shown in FIGS. 1 and 2, the optical signal 4 is generated by a transmitter 6. The transmitter 6 is located structurally united with the object platform 2.

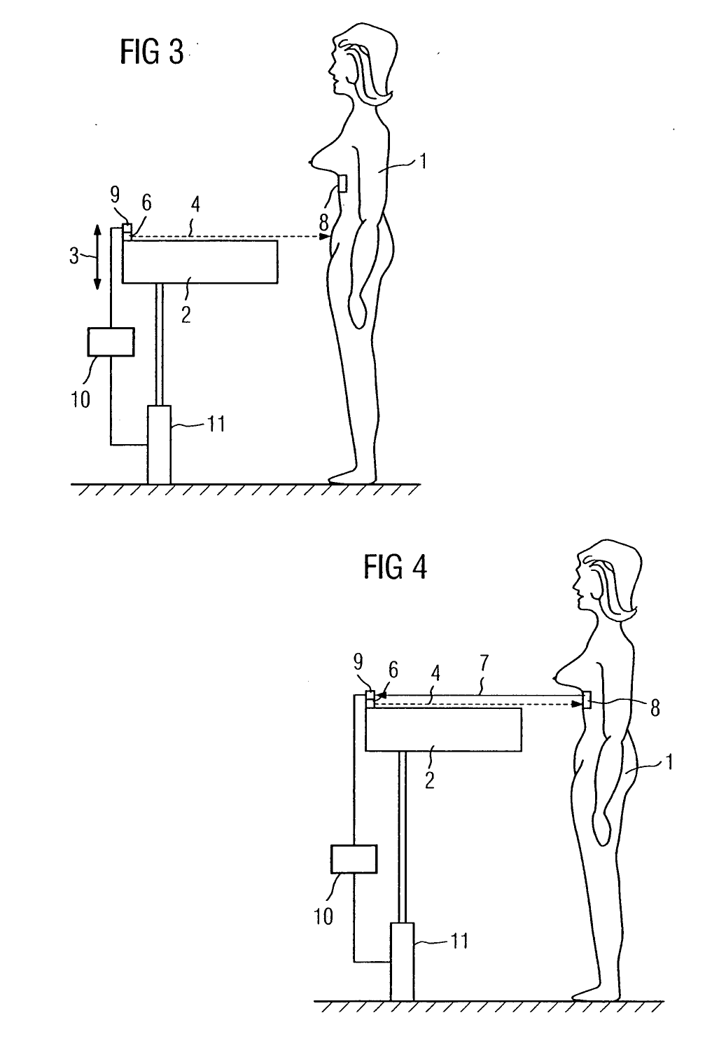

In one embodiment, as shown in FIGS. 3 and 4, a reflector 8 is provided directly below the examination region. The projected optical signal 4 is reflected from the patient 1 once the object platform 2, in its height adjustment, has reached the height corresponding to the examination region of the patient 1. The projected optical signal 4 is reflected by the reflector 8.

As shown in FIG. 3, an optical signal 4 is projected in the direction of the patient 1 by a transmitter 6 that is structurally combined with the object platform 2. The optical signal 4 strikes the patient 1 below the reflector 8 and is not reflected to the object platform 2. The height of the object platform 2 is adjustable in the direction of the arrow 3 shown in FIG. 3. As shown in FIG. 4, when the height adjustment of the object platform 2 that is suitable for the examination is reached, the projected optical signal 4 strikes the reflector 8 and is reflected. The reflected optical signal 7 is recognizable to the mammography device operator.

In one embodiment, a sensor 9, in particular structurally united with the object platform 2, is provided. The sensor 9 indicates the reflected optical signal 7 by tripping an optical and/or acoustical signal. The sensor 9 makes signal detection possible when a non-visible optical signal 4 or 7 is employed, such as infrared light.

In one embodiment, the reflected signal 7 measured by the sensor 9 serves as a criterion for a control unit 10, which automatically adjusts the height of the object platform 2 with a vertical drive mechanism 11.

One embodiment can be summarized as follows:

A mammography device for adjusting the height of an object platform 2 relative to the examination region of a patient 1 includes an optical signal 4. The height of the object platform 2 be reproduced on the patient. The adjusted height of the object platform 2 is varied until the optical signal 4 is reflected onto the object platform 2 by a reflector 8 located directly below the examination region. In one embodiment, the reflected signal is detected by a sensor 9 and used for indicating a tripped optical and/or acoustical signal. The sensor 9 may also trigger a vertical drive mechanism 11.

1. A mammography device comprising:
an adjustable-height platform, and
a transmitter that generates an optical signal,
the optical signal being dependent on the height of the platform and being projectable onto a patient standing in front of the platform.

2. The mammography device as defined by claim 1, wherein the projected optical signal reproduces the height of the object platform on the patient.

3. A mammography device comprising:
an adjustable-height object platform, and
a transmitter operable to generate an optical signal, the optical signal being dependent on the height of the object platform and being projectable onto a patient standing in front of the object platform,
wherein the optical signal is reflectable from the patient when the height of an examination area coincides with the height of the object platform.

4. The mammography device as defined by claim 3, having a reflector that reflects the optical signal, the reflector being disposed directly below the examination region of the patient.

5. The mammography device as defined by claim 3, a sensor that responds to the reflected optical signal, the sensor being coupled with the mammography device or the object platform.

6. The mammography device as defined by claim 5, wherein the height of the object platform is automatically adjusted as a function of the reflected signal measured by the sensor.

7. The mammography device as defined by claim 1, wherein the transmitter is coupled with the mammography device or the object platform.

8. The mammography device as defined by claim 1, wherein the optical signal includes a beam of light.

9. The mammography device as defined claim 1, wherein the optical signal includes a laser beam.

10. The mammography device as defined by claim 4, comprising a sensor that responds to the reflected optical signal, the sensor being coupled with the mammography device or the object platform.

11. The mammography device as defined by claim 10, wherein the height of the object platform is adjusted as a function of the reflected signal measured by the sensor is provided.

12. The mammography device as defined by claim 6, wherein the transmitter is coupled with the mammography device or the object platform.

13. The mammography device as defined by claim 11, wherein the transmitter is coupled with the mammography device or the object platform.

14. The mammography device as defined by claim 12, wherein the optical signal includes a beam of light.

15. The mammography device as defined by claim 13, wherein the optical signal includes a beam of light.

16. The mammography device as defined claim 12, wherein the optical signal includes a laser beam.

17. The mammography device as defined claim 13, wherein the optical signal includes a laser beam.
